(19) United States
(12) Patent Application Publication
(10) Pub. No.: US 2018/0021404 A1
(43) Pub. Date: Jan. 25, 2018

(54) METHODS AND COMPOSITIONS FOR TREATING GLAUCOMA

(71) Applicant: The Regents of the University of California, Oakland, CA (US)

(72) Inventors: Jeffrey L. Goldberg, La Jolla, CA (US); Travis L. Stiles, La Jolla, CA (US)

(21) Appl. No.: 15/551,205

(22) PCT Filed: Feb. 19, 2016
(86) PCT No.: PCT/US16/18607

§ 371 Date: Aug. 15, 2017

Related U.S. Application Data
(60) Provisional application No. 62/118,642, filed on Feb. 20, 2015.

(51) Int. Cl. A61K 38/17; A61K 38/18; C12N 7/00; A61K 9/00

(52) U.S. Cl. CPC A61K 38/177; A61K 9/0051; A61K 38/185; C12N 7/00; C12N 2710/10043; C12N 2750/14143; C12N 2710/16643; C12N 2740/15043

(57) ABSTRACT

Provided are methods and compositions for treating an ocular disease associated with increased intraocular pressure using a therapeutically effective amount of receptor associated protein (RAP), a derivative of RAP, a variant of RAP, or a fragment of RAP. Also provided are methods and compositions for reducing intraocular pressure.

### CROSS-REFERENCE TO RELATED APPLICATION

This application claims priority to U.S. Provisional Application No. 62/118,642, filed on Feb. 20, 2015, which is incorporated herein by reference in its entirety.

### FIELD OF THE INVENTION

The present invention relates to treatments for ocular diseases, including ocular hypertension and glaucoma. The treatments use the biologic RAP (receptor associated protein), derivatives/variants of RAP, and/or fragments of RAP for reducing intraocular pressure (IOP) of the eye.

### BACKGROUND INFORMATION

Glaucoma is characterized by a permanent loss of visual function due to irreversible damage to the optic nerve. There are several morphologically or functionally distinct types of glaucoma. All types are characterized by elevated intraocular pressure (IOP), which is considered to be causally related to the pathological course of the disease. Ocular hypertension is a condition wherein intraocular pressure is elevated, but no apparent loss of visual function has occurred; such patients are considered to be at high risk for the eventual development of the visual loss associated with glaucoma. If glaucoma or ocular hypertension is detected early and treated promptly, loss of visual function or the progressive deterioration thereof can often be ameliorated. A general method of treating glaucoma is intraocular pressure reduction therapy, which is exemplified by pharmacotherapy, laser therapy, and surgery.

Drug therapies that are effective for the treatment of glaucoma and ocular hypertension include agents that reduce intraocular pressure. Such therapies are, in general, administered by one of two possible routes: topically (direct application to the eye) or orally. However, pharmaceutical ocular anti-hypertension approaches have exhibited various undesirable side effects. For example, miotics such as pilocarpine can cause blurring of vision, headaches, and other negative visual side effects. Systemically administered carbonic anhydrase inhibitors can cause nausea, dyspepsia, fatigue, and metabolic acidosis. Certain prostaglandins cause hyperemia, ocular itching, and darkening of eyelashes and periorbital skin. Such negative side-effects may lead to decreased patient compliance or to termination of therapy such that normal vision continues to deteriorate. Additionally, there are individuals who simply do not respond well when treated with certain existing glaucoma therapies.

Current clinically approved therapeutics for glaucoma are primarily directed at decreasing intraocular pressure, either through decreasing vitreous production or increasing fluid outflow using chemical drugs or surgery/surgical implants. Depending on the type of glaucoma, surgical interventions are sometimes utilized to improve drainage from the eye. However, the majority of glaucoma cases are managed medically via the following mechanisms: alpha agonists (decrease fluid, increase drainage) drops, beta blocker (decrease fluid production) drops, carbonic anhydrase inhibitors (decrease fluid production) drops/pills, cholinergic (increased drainage through meshwork), prostaglandin analogs (increased outflow) drops. All of these latter drugs are chemical (not biological) and thus cannot be delivered with cells or viral delivery systems. There is, therefore, a need for other therapeutic agents for the treatment of glaucoma and ocular hypertension including biologic therapeutic agents.

### SUMMARY OF THE INVENTION

The present invention is based on the finding that disruption of the function of the low-density lipoprotein receptor-related protein 1 (LRP1) has the ability to decrease Rho activity. Inhibition of Rho in the cells within the trabecular meshwork of the eye has been shown to increase vitreous outflow and reduce IOP. Receptor-associated protein (RAP) is a potent, broad spectrum low-density lipoprotein receptor (LDLR) family antagonist that has the ability to reduce IOP when injected into the eye, without being bound by theory, due to antagonism of LRP1.

Accordingly, the present invention provides a method of treating an ocular disease comprising administering to a subject in need thereof a therapeutically effective amount of RAP or a derivative/variant or fragment thereof, thereby treating the ocular disease. As appropriate, RAP or a derivative/variant or fragment thereof can be delivered to the eye as a polypeptide (or variants or fragments thereof), or as a polynucleotide encoding RAP (e.g., in a plasmid or viral vector) or a derivative/variant or fragment thereof. The disease may be any ocular disease associated with increased ocular pressure, such as glaucoma and intraocular hypertension. The RAP or a derivative/variant or fragment thereof may be administered via intravitreal injection and may result in increased aqueous outflow of the vitreous humor of the eye. The subject may be a mammal, such as a human. The administration can include intravitreal delivery of a pharmaceutical composition that comprises a delivery vehicle and an expression vector that encodes the RAP, derivative of RAP, variant of RAP, or fragment of RAP. The expression vector may be an adenovirus vector, an adeno-associated virus vector, a herpes simplex virus-based vector, a lentivirus vector, or a plasmid vector.

In another aspect, the invention provides a method of treating glaucoma comprising administering to a subject in need thereof a therapeutically effective amount of receptor associated protein (RAP) or a derivative/variant or fragment thereof, thereby treating glaucoma. The RAP or a derivative/variant or fragment thereof may be administered via intravitreal injection and may result in increased aqueous outflow of the vitreous humor of the eye. The subject may be a mammal, such as a human. In various embodiments, the administration may include intravitreal delivery of a pharmaceutical composition that comprises a delivery vehicle and an expression vector that encodes the RAP, derivative of RAP, variant of RAP, or fragment of RAP. The expression vector may be an adenovirus vector, an adeno-associated virus vector, a herpes simplex virus-based vector, a lentivirus vector, or a plasmid vector.

In another aspect, the invention provides a method of reducing intraocular pressure in an eye of a subject comprising administering a RAP-conjugated agent, a RAP derivative-conjugated agent, a RAP variant-conjugated agent, or a RAP fragment-conjugated agent. In various embodiments, the agent can be a neurotrophic or neuroprotective agent, such as brain-derived neurotrophic factor (BDNF), nerve growth factor (NGF), neurotrophin-3/4 (NT-3/4), and ciliary neurotrophic factor (CNTF). The RAP-conjugated agent, the RAP derivative-conjugated agent, the RAP variant-conjugated agent, or the RAP fragment-conjugated agent can be administered via intravitreal injection and may result in increased aqueous outflow of the vitreous humor of the eye. The subject may be a mammal, such as a human. In various embodiments, the administration may include intravitreal delivery of a pharmaceutical composition that comprises a delivery vehicle and an expression vector that encodes the RAP-conjugated agent, the RAP derivative-conjugated agent, the RAP variant-conjugated agent, or the RAP fragment-conjugated agent. The expression vector may be an adenovirus vector, an adeno-associated virus vector, a herpes simplex virus-based vector, a lentivirus vector, or a plasmid vector.

The methods disclosed herein can also be practiced using an inhibitor of LRP1, such as an inhibitory nucleic acid that inhibits the expression of LRP1. For example, the inhibitory nucleic acid that inhibits the expression of LRP1 can be a siRNA, a shRNA, an antisense RNA or a ribozyme. As appropriate, the inhibitory nucleic acid can be delivered in a viral vector, for example, a neurotropic viral vector. In some embodiments, the inhibitor of LRP1 is a siRNA or shRNA that specifically inhibits the expression of LRP1. As appropriate, the siRNA or shRNA can be delivered in a lentiviral vector, a herpesvirus vector or an adenoviral vector.

The present invention also provides uses of the compounds (e.g., RAP or a derivative/variant or a fragment thereof) of the invention and/or compositions of the invention for treating ocular diseases, and for reducing ocular IOP. The ocular disease can be glaucoma and/or intraocular hypertension. The present invention also provides uses of the compounds (e.g., RAP or a derivative/variant or fragment thereof) of the invention and/or compositions of the invention in the manufacture of a medicament for treating ocular diseases, and for reducing IOP. The ocular disease can be glaucoma and/or intraocular hypertension.

### BRIEF DESCRIPTION OF THE DRAWINGS

FIG. 1 is a graph showing the results of RAP as a therapeutic for the lowering of intraocular pressure.

### DETAILED DESCRIPTION

The present disclosure is based on the observation that intraocular pressure of the eye can be reduced via administration of the biologic RAP (receptor associated protein) or a derivative/variant or fragment thereof. U.S. Pat. Nos. 5,798,380, 6,110,912, and 6,586,425, U.S. Pub. No. 2012/0040994, and international Pub. No. WO 2004/079829, the entirety of each of which is incorporated herein by reference, describe in detail the nature and etiology of glaucoma and various therapeutic approaches for reducing intraocular pressure characteristic of the disorder.

RAP is a molecular chaperone for members of the low-density lipoprotein receptor (LDLR) family. RAP binds tightly to newly synthesized LDLR members in the endoplasmic reticulum and facilitates their delivery to the Golgi apparatus. The nucleic acid and polypeptide sequences of RAP in humans are available at the website for the National Center for Biotechnology Information as GenBank Accession No. NM_002337.2 and GenBank Accession No. NP_002328, respectively.

RAP is a potent, broad spectrum low-density lipoprotein receptor (LDLR) family antagonist, including LRP1. Antagonism of LRP1 blocks pathological activation of RhoA. Inhibition of Rho in the cells within the trabecular meshwork of the eye changes the cytoskeletal contractility of these cells, resulting in increased vitreous outflow from the eve. Increased outflow results in decreased intraocular pressure. RAP reduces IOP when injected into the eye, without being bound by theory, due to antagonism of LRP1 resulting in Rho inactivation. In cell culture studies, RAP concentrations used to achieve maximal LRP1 antagonism are most commonly found between 100-500 nM, although these concentrations are well in excess of the concentration needed to achieve maximal receptor occupancy.

RAP is actively transported into the brain across the blood-brain barrier, giving it a practical advantage in treatment of RhoA pathology in the central nervous system. The ability of RAP to block activation of RhoA is limited to signaling mechanisms inherent to pathological activation (via distinct signaling complexes) and does not interfere with the important endogenous activities important for basal cell functions such as migration and cell division. Additionally, RAP acts on surface receptor complexes, thereby bypassing the need for conjugation to cell-permeabilization tags. For these reasons, RAP is superior to other biologic RhoA inhibitors, such as C3 transferase, which can indiscriminately block RhoA activity and requires a tat-fusion motif, which is a major contributor to HIV dementia, thereby raising questions of long-term side effects. The biologic RAP and derivatives/variants and fragments thereof have superior capacity to reduce intraocular pressure in rodents given intraocular injections of each agent. As such, RAP provides a number of favorable characteristics that indicate greater therapeutic potential for glaucoma, intraocular hypertension, and other diseases associated with elevated IOP over current biologics, such as C3 transferase.

Accordingly, the present disclosure provides compositions and methods for reducing intraocular pressure in a subject in need thereof by administering RAP. In various embodiments, the subject can be suffering from glaucoma or any disease having signs or symptoms related to increased intraocular pressure.

The terms “low density lipoprotein receptor-related protein associated protein 1,” “LRPAP1,” “alpha-2-macroglobulin receptor-associated protein,” “receptor associated protein,” and “RAP” interchangeably refer to nucleic acids and polypeptide polymorphic variants, alleles, mutants, and interspecies homologs that: (1) have an amino acid sequence that has greater than about 90% amino acid sequence identity, for example, 91%, 92%, 93%, 94%, 95%, 96%, 97%, 98% or 99% or greater amino acid sequence identity, preferably over a region of at least about 25, 50, 100, 200, 300, 400, or more amino acids, or over the full-length, to an amino acid sequence encoded by a RAP nucleic acid (see, e.g., GenBank Accession No. NM_002337.2) or to an amino acid sequence of a RAP polypeptide (see. e.g., GenBank Accession No. NP_002328.1); (2) bind to antibodies, e.g., polyclonal antibodies, raised against an immunogen comprising an amino acid sequence of a RAP polypeptide (e.g., RAP polypeptides described herein); or an amino acid sequence encoded by a RAP nucleic acid (e.g., RAP polynucleotides described herein), and conservatively modified variants thereof; (3) specifically hybridize under stringent hybridization conditions to an anti-sense strand corresponding to a nucleic acid sequence encoding a RAP protein, and conservatively modified variants thereof; or (4) have a nucleic acid sequence that has greater than about 90%, preferably greater than about 91%, 92%, 93%, 94%, 95%, 96%, 97%, 98%, 99%, or higher nucleotide sequence identity, preferably over a region of at least about 25, 50, 100, 200, 500, 1000, 2000 or more nucleotides, or over the full-length, to a RAP nucleic acid (e.g., RAP polynucleotides, as described herein, and RAP polynucleotides that encode RAP polypeptides, as described herein).

Exemplary RAP derivatives/variants useful in the methods of the present invention include, but are not limited to, those compounds disclosed in U.S. Pat. Nos. 8,440,629, 8,609,103, and 7,560,431; in U.S. Pub. Nos. 2009/0291024 and 2009/0269346; and in international publication nos. WO 2008/036682 and WO 2005/002515, the entire content of each of which is incorporated herein by reference. A RAP fragment is a portion of RAP that still retains some, substantially all, or all of the therapeutic or biological activity of RAP. For example, RAP fragments useful in the present methods are portions of RAP which retain the therapeutic or biological activity of RAP to reduce activation of Rho, to treat ocular diseases, for example glaucoma and intraocular hypertension, and/or to reduce IOP.

The use of biologics, such as RAP, in the lowering of intraocular pressure has several advantages over existing glaucoma drugs in that the method of delivery can be expanded beyond the application of topical drops or systemically delivered oral drug, including long-term expression of the biological inside the eye. Delivery mechanisms for RAP in the methods described herein can include viral and cell-based delivery systems, intravitreal delivery, e.g., intravitreal injection, gene transfer, nanoparticles, and implantable meshes. Intravitreal injection can be particularly effective in the disclosed methods. Other effective mechanisms include sustained delivery mechanisms, which remove the need for repeated administration and restricts drug delivery to a target tissue, thereby reducing potential off-target effects and toxicity. The eye is suitable for such approaches as the self-contained nature of the intraocular space can restrict leakage and immune infiltration that can lead to adverse immune activity and inflammation.

In the present disclosure, RAP can be formulated in a standard phosphate buffered saline solution at a pH of 7.3 without carrier protein. However, the present disclosure is not limited to this formulation of RAP.

High concentration RAP solutions undergoing a single freeze/thaw cycle in PBS consistently demonstrate less than 5% degradation or loss of product, indicating that RAP maintains its solubility even in the presence of pH changes inherent in the freezing process (upwards of 1.0 pH unit from the desired neutral pH of 7.3). Degradation of RAP in standard PBS solution at a concentration of 200 μM is routinely observed to be negligible after 2 weeks at 4 degrees, and only approximately 5-10% degradation can be observed via SDS-PAGE analysis at 2 weeks of storage at room temperature. RAP solution stored at −80° C. appears to suffer negligible degradation up to 1-year after freeze. However, upon multiple freeze/thaw cycles it is not uncommon to observe RAP degradation between 20-50%, as observed by SDS-PAGE analysis, and as such, handling in this manner is not recommended. Degradation of RAP at 37° is consistently negligible for at least 5 days. RAP quality control consists of identity confirmation via SDS-PAGE and mass spectrometry, and internally defined functional confirmation based on the ability of RAP to bind and antagonize LRP1.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

As used herein, the term “intraocular pressure” or “IOP” refers to the pressure of the fluid inside the eye. This pressure varies among individuals, for example, IOP may become elevated due to anatomical problems, inflammation of the eye, as a side-effect from medication or due to genetic factors. Elevated intraocular pressure is a significant risk factor for glaucoma.

The term “subject” as used herein refers to any individual or patient to which the subject methods are performed. Generally the subject is human, although as will be appreciated by those in the art, the subject may be an animal. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of the term “subject.”

The invention provides a method of treating glaucoma in a subject. The method includes administering RAP or a derivative/variant or fragment thereof to the eye of the subject, thereby reducing intraocular pressure in the eye and treating glaucoma. As used herein, the term “ameliorating” or “treating” means that the clinical signs and/or the symptoms associated with an ocular disorder (e.g., glaucoma) are lessened as a result of the actions performed. The signs or symptoms for monitoring are characteristic of the ocular disorder (e.g., glaucoma) and are well known to the skilled clinician, as are methods for monitoring the signs and conditions. For example, subjects having an ocular disorder may experience fuzzy or blurry vision, an empty or dark area in the center of vision, the appearance of straight lines, such as sides of buildings, telephone poles, or sentences on a page, as curved or wavy, and/or a dimming, of vision when reading. Also included in the definition of “ameliorating” or “treating” is the lessening of symptoms associated with the ocular disorders in subjects not yet diagnosed as having the specific disorders. As such, the methods may be used as a means for prophylactic therapy for a subject at risk of having a specific ocular disorder.

The signs and symptoms associated with an ocular disorder (e.g., glaucoma) can be monitored by assessment via Optical Coherence Tomography (OCT). OCT is a non-invasive, fast, non-contact imaging technique which readily displays intra-retinal, subretinal and sub-RPE (retinal pigment epithelium) fluid. OCT relies upon differential reflections of light to produce 2-dimensional cross-sections of the retina. OCT images are obtained rapidly and have a spatial resolution of approximately 8 mcm. OCT is especially useful for calculating retinal thickness. In another embodiment, the signs and symptoms associated with an ocular disorder (e,g., glaucoma) may be monitored by assessment via a visual acuity (VA) test. The visual acuity test is used to determine the smallest letters a person can read on a standardized chart or card held 14-20 feet away. This test is done on each eye, one at a time. If necessary, it is then repeated while the subject wears glasses or contacts.

As used herein, the terms “reduce” and “inhibit” are used together because it is recognized that, in some cases, a decrease, for example, in Rho activity can be reduced below the level of detection of a particular assay. As such, it may not always be clear whether the activity is “reduced” below a level of detection of an assay, or is completely “inhibited”. Nevertheless, it will be determinable, following a treatment according to the present methods, that the level of Rho activity and/or the level of intraocular pressure and/or the level of LRP1 expression in the particular region or cells being tested is at least reduced from the level before treatment.

In one embodiment, the methods provided herein for treating an ocular disease associated with increased intraocular pressure includes administering to the subject in need thereof a therapeutically effect amount of RAP, a derivative/variant of RAP, a fragment of RAP, and/or an inhibitor of LRP1 activity. The term “effective amount” or “therapeutically effective amount” refers to the amount of an active agent sufficient to induce a desired biological result (e.g., reduction in intraocular pressure either through decreasing vitreous production or increasing fluid outflow, promotion and/or restoration of neuronal regeneration and/or neurite growth). That result may be alleviation of the signs, symptoms, or causes of a disease, or any other desired alteration of a biological system. The term “therapeutically effective amount” is used herein to denote any amount of the formulation which causes a substantial improvement in a disease condition when applied to the affected areas repeatedly over a period of time. The amount will vary with the condition being treated, the stage of advancement of the condition, and the type and concentration of formulation applied. Appropriate amounts in any given instance will be readily apparent to those skilled in the art or capable of determination by routine experimentation.

A “therapeutic effect,” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, I-carboxyglutamate, and O-phosphoserine. Amino acid analogs refers to compounds that have the same basic chemical structure as a naturally occurring amino acid, i.e., an I carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W); and
7) Serine (S), Threonine (T)
   (see, e.g., Creighton, Proteins (1984)).

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, e.g., a RAP polynucleotide or polypeptide sequence, a derivative/variant thereof, or fragment thereof as described herein, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, for example, over a region that is 50-100 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to RAP nucleic acids and proteins (and/or derivatives/variants thereof), the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., eds., Current Protocols in Molecular Biology (1995 supplement)).

Examples of algorithms that are suitable for determining percent sequence identity and sequence similarity are the BLAST and BLAST 2.0 algorithms, which are described in Altschul et al., J. Mol. Biol. 215:403-410 (1990) and Altschul et al., Nucleic Acids Res. 25:3389-3402 (1977), respectively. Software for performing BLAST analyses is publicly available through the National Center for Biotechnology Information (on the worldwide web at ncbi.nlm.nih.gov/). The algorithm involves first identifying high scoring sequence pairs (HSPS) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPS containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “antibody” as used herein refers to polyclonal and monoclonal antibodies and fragments thereof, and immunologic binding equivalents thereof. The term “antibody” refers to a homogeneous molecular entity, or a mixture such as a polyclonal serum product made up of a plurality of different molecular entities, and broadly encompasses naturally-occurring forms of antibodies (for example, IgG, IgA, IgM, IgE) and recombinant antibodies such as single-chain antibodies, chimeric and humanized antibodies and multi-specific antibodies. The term “antibody” also refers to fragments and derivatives of all of the foregoing, and may further comprise any modified or derivatised variants thereof that retains the ability to specifically bind an epitope. Antibody derivatives may comprise a protein or chemical moiety conjugated to an antibody. A monoclonal antibody is capable of selectively binding to a target antigen or epitope. Antibodies may include, but are not limited to polyclonal antibodies, monoclonal antibodies (mAbs), humanized or chimeric antibodies, camelized antibodies, single chain antibodies (scFvs), Fab fragments, F(ab′)2 fragments, disulfide-linked Fvs (sdFv) fragments, for example, as produced by a Fab expression library, anti-idiotypic (anti-Id) antibodies, intrabodies, nanobodies, synthetic antibodies, and epitope-binding fragments of any of the above.

The terms “administration” or “administering” are defined to include the act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravitreal, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection, intracameral and intravitreal injection, and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

Thus, the compounds of the invention can be administered in any way typical of an agent used to treat the particular type of ocular disorder, or under conditions that facilitate contact of the agent with target intraocular cells and, if appropriate, entry into the cells. Entry of a polynucleotide agent into a cell, for example, can be facilitated by incorporating the polynucleotide into a viral vector that can infect the cells. Specific examples of such approaches include, but are not limited to, lenti or adenoviral derived expression systems for RAP or shRNA against LRP1, stable-expressing and secreting cell-delivery systems capable of long-term release of RAP (or similar agents such as receptor decoys), or bioavailable topical solutions capable of administration in drop form.

If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide agent can be introduced into a cell by various methods, including, for example, by engineering the peptide to contain a protein transduction domain such as the human immunodeficiency virus TAT protein transduction domain, which can facilitate translocation of the peptide into the cell. In addition, there are a variety of biomaterial-based technologies such as nano-cages and pharmacological delivery wafers (such as used in brain cancer chemotherapeutics) which may also be modified to accommodate this technology.

Methods for chemically modifying polynucleotides and polypeptides, for example, to render them less susceptible to degradation by endogenous nucleases or proteases, respectively, or more absorbable through the alimentary tract are well known (see, for example, Blondelle et al., Trends Anal. Chem. 14:83-92, 1995; Ecker and Crook, BioTechnology, 13:351-360, 1995). For example, a peptide agent can be prepared using D-amino acids, or can contain one or more domains based on peptidomimetics, which are organic molecules that mimic the structure of peptide domain; or based on a peptoid such as a vinylogous peptoid. Where the compound is a small organic molecule such as a steroidal alkaloid, it can be administered in a form that releases the active agent at the desired position in the body (e.g., the eye), or by injection into a blood vessel such that the inhibitor circulates to the target cells (e.g., intraocular cells).

The compounds of the invention are also suitably administered by sustained-release systems. Suitable examples of sustained-release compositions include, but are not limited to, semi-permeable polymer matrices in the form of shaped articles, e.g., films, or mirocapsules. Sustained-release matrices include polylactides (U.S. Pat No. 3,773,919, EP 58,481 incorporated herein by reference), copolymers of L-glutamic acid and gamma-ethyl-L-glutamate (U. Sidman et al., Biopolymers 22:547-556 (1983)), poly (2-hydroxyethyl methacrylate) (R. Langer et al., J. Biomed Mater. Res. 15:167-277 (1981), and R. Langer, Chem. Tech. 12:98-105 (1982)), ethylene vinyl acetate (R. Langer et al., Id.) or poly-D-(−)-3-hydroxy butyric acid (EP 133,988). Liposomes containing the compounds of the invention may be prepared by methods known in the art: Epstein et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal delivery of the compounds of the invention.

In certain embodiments, the invention compounds may further be administered (i.e., co-administered) in combination with an antiinflammatory, antimicrobial, antihistamine, chemotherapeutic agent, antiangiogenic agent, immunomodulator, therapeutic antibody or a neuroprotective agent, to a subject in need of such treatment. Other agents that may be administered in combination with invention compounds include protein therapeutic agents such as cytokines, immunomodulatory agents and antibodies. While not wanting to be limiting, antimicrobial agents include antivirals, antibiotics, anti-fungals and anti-parasitics. When other therapeutic agents are employed in combination with the inhibitors of the present invention they may be used for example in amounts as noted in the Physician Desk Reference (PDR) or as otherwise determined by one having ordinary skill in the art.

The term “co-administer” and “co-administering” and variants thereof refer to the simultaneous presence of two or more active agents in an individual. The active agents that are co-administered can be concurrently or sequentially delivered. As used herein, RAP can be co-administered with another active agent efficacious in promoting neuronal regeneration in the CNS.

Conceptually, any substance capable of interfering with ligand binding to LRP1, or competitively displacing ligands responsible for induction of undesirable receptor function, would be therapeutically similar to RAP and/or may be used in conjunction with RAP. Since we are unaware of the specific molecular interactions with LRP1 responsible for LRP1-mediated Rho activity, it is possible that all LRP1 ligands are potentially capable of interfering with Rho. However, LRP1 has been demonstrated to be capable of diverse effects on other GTPases such as Rac. Thus, exemplary LRP1 ligands having Rho-impairing capacity include, but are not limited to, lactoferrin, isoforms of apolipoprotein-E (ApoE), inactivated forms of alpha-2-macroglobulin, lipoprotein lipase, hepatic lipase, amyloid precursor protein (APP), and various heat shock proteins. These ligands are specifically selected due to their distinction from ligands identified in the literature as “signaling” ligands.

Myelin-associated glycoprotein (MAG) is a component of myelin that has been shown to induce Rho activation in neurons in culture at least in part due to LRP1. Incubation of myelin with recombinant derivatives of LRP1 that reconstitute the binding domain for MAG is comparable to RAP in overcoming the effects of myelin, including activation of Rho. Without being bound by theory, LRP1 has been shown to be constitutively trafficked from the cell surface into endosomes and back, due at least in part to its diverse ligand binding and endocytic capacity. While it is possible that RAP and derivatives thereof impairs basal LRP1-mediated activation of RhoA, it is equally as plausible that the effects of LRP1 on Rho can be attributed to trafficking of ligand. Similar to the receptor decoys used to overcome the effects of myelin, it is possible that identifying LRP1 domains that are capable of binding ligands responsible for LRP1-mediated activation of Rho may be of similar therapeutic use as RAP. As such, the invention provides methods of screening for truncated versions of LRP1 or small molecules for use as soluble receptor decoys.

In some embodiments, the candidate agent is a small organic compound, a polypeptide, an antibody or fragment thereof, an amino acid or analog thereof, a carbohydrate, a saccharide or disaccharide, or a polynucleotide. Thus, small molecule compounds may be selected to inhibit LRP1 expression or activity in the eye. This can include, but is not limited to, molecules that interfere with LRP1 ligand binding, modify or block the trafficking/translocation of LRP1, or cleave the extracellular domain of LRP1 from the cell surface. Such cleavage may be uniquely accomplished via the use of specific gamma-secretases (i.e., ADAM17 or ADAM10) or by exploiting the furin cleavage site used in the initial processing of the receptor. In addition, interfering RNA (RNAi) technology capable of targeting trabecular meshwork cells could also be of use.

RNAi is a phenomenon in which the introduction of dsRNA into a diverse range of organisms and cell types causes degradation of the complementary mRNA. In the cell, long dsRNAs are cleaved into short (e.g., 21-25 nucleotide) small interfering RNAs (siRNAs), by a ribonuclease. The siRNAs subsequently assemble with protein components into an RNA-induced silencing complex (RISC), unwinding in the process. The activated RISC then binds to complementary transcripts by base pairing interactions between the siRNA antisense strand and the mRNA. The bound mRNA is then cleaved and sequence specific degradation of mRNA results in gene silencing. As used herein, “silencing” refers to a mechanism by which cells shut down large sections of chromosomal DNA resulting in suppressing the expression of a particular gene. Without being bound by theory, the RNAi machinery appears to have evolved to protect the genome from endogenous transposable elements and from viral infections. Thus, RNAi can be induced by introducing nucleic acid molecules complementary to the target mRNA to be degraded, as described herein.

The screening methods of the invention can be conveniently carried out using high-throughput methods. In some embodiments, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int J Pept Prot Res 37:487-493 (1991) and Floughton, et al., Nature 354:84-88 (1991)). Other chemistries for generating chemical diversity libraries can also be used. Such chemistries include, but are not limited to peptoids (e.g., PCT Publication No. WO 91/19735), encoded peptides (e.g., PCT Publication WO 93/20242), random bio-oligomers (e.g., PCT Publication No WO 92/00091), benzodiazepines (e.g., U.S. Pat No. 5,288,514), diversomers such as hydantoins, benzodiazepines and dipeptides (Hobbs et al, Proc Nat Acad Sci USA 90:6909-6913 (1993)), vinylogous polypeptides (Hagihara, et al., J Amer Chem Soc 114:6568 (1992)), nonpeptidal peptidomimetics with glucose scaffolding (Hirschmann et al., J Amer Chem Soc 114:9217-9218 (1992)), analogous organic syntheses of small compound libraries (Chen et al., J Amer Chem Soc 116:2661 (1994)), oligocarbamates (Cho et al., Science 261:1303 (1993)) and/or peptidyl phosphonates (Campbell et al., J Org Chem 59:658 (1994)), nucleic acid libraries, peptide nucleic acid libraries (see, e.g., U.S. Pat. No. 5,539,083), antibody libraries (see, e.g., Vaughn et al., Nature Biotechnology, 14(3):309314 (1996) and PCT/US96/10287), carbohydrate libraries (see, e.g., Liang et al., Science 274:1520-1522 (1996) and U.S. Pat. No. 5,593,853), small organic molecule libraries (see, e.g., benzodiazepines, Baum, C&EN, January 18, page 33 (1993), isoprenoids, U.S. Pat. No. 5,569,588), thiazolidinones and metathiazanones, U.S. Pat. No. 5,549,974 pyrrolidines, U.S. Pat. Nos. 5,525,735 and 5,519,134, morpholino compounds, U.S. Pat. No. 5,506,337 benzodiazepines, U.S. Pat. No. 5,288,514, and the like).

In high throughput assays of the invention, it is possible to screen up to several thousand different candidate agents in a single day. In particular, each well of a microtiter plate can be used to run a separate assay against a selected potential candidate agent, or, if concentration or incubation time effects are to be observed, every 5-10 wells can test a single modulator. Thus, a single standard microtiter plate can assay about 100 (e.g., 96) candidate agents. Multiwell plates with greater numbers of wells find use, e.g., 192, 384, 768 or 1536 wells. If 1536-well plates are used, then a single plate can easily assay from about 100 to about 1500 different compounds. It is possible to assay several different plates per day. Assay screens for up to about 6,000-20,000 different compounds are possible using the integrated systems of the invention.

In certain embodiments, the invention compositions may include RAP-conjugated neurotrophic or neuroprotective agents. Exemplary neurotrophic or neuroprotective agents include, but are not limited to, neurotrophins (e.g., brain-derived neurotrophic factor (BDNF), nerve growth factor (NGF), neurotrophin-3/4 (NT-3/4), ciliary neurotrophic factor (CNTF)), cyclic nucleotide homologs (e.g., cAMP derivatives), C3 transferase derivatives, stimulators of adenylyl cyclases (e.g., forskolin and other hormones).

All the methods and compositions in PCT/US2012/035125 (publication #WO 2012/149111 A1) are hereby incorporated by reference in their entireties.

In certain embodiments, the compositions for use in the methods of the present invention further comprise a targeting moiety. Targeting moieties include a protein or a peptide which directs localization to a certain part of the body, for example, to the brain or spine, or compartments therein. In certain embodiments, compositions for use in the methods of the present invention are attached or fused to a brain targeting moiety. The brain targeting moieties are attached covalently (e.g., direct, translational fusion, or by chemical linkage either directly or through a spacer molecule, which can be optionally cleavable) or non-covalently attached (e.g., through reversible interactions such as avidin:biotin, protein A:IgG, etc.). In other embodiments, the compounds for use in the methods of the present invention thereof are attached to one more brain targeting moieties. In additional embodiments, the brain targeting moiety is attached to a plurality of compounds for use in the methods of the present invention

A CNS targeting moiety associated with a compound enhances CNS delivery of such compositions. A number of polypeptides have been described which, when fused to a therapeutic agent, delivers the therapeutic agent through the blood brain barrier (BBB). Nonlimiting examples include the single domain antibody FC5 (Abulrob et al. (2005) J. Neurochem. 95, 1201-1214); mAB 83-14, a monoclonal antibody to the human insulin receptor (Pardridge et al. (1995) Pharmacol. Res. 12, 807-816); the B2, B6 and B8 peptides binding to the human transferrin receptor (hTfR) (Xia et al. (2000) J. Virol. 74, 1135911366); the OX26 monoclonal antibody to the transferrin receptor (Pardridge et al. (1991) J. Pharmacol. Exp. Ther. 259, 66-70); diptheria toxin conjugates. (see, for e.g., Gaillard et al., International Congress Series 1277:185-198 (2005); and SEQ ID NOs: 1-18 of U.S. Pat. No. 6,306,365. The contents of the above references are incorporated herein by reference in their entirety).

The methods of the invention are also useful for providing a means for practicing personalized medicine, wherein treatment is tailored to a subject based on the particular characteristics of the ocular disorder from which the subject is suffering. The method can be practiced, for example, by contacting a sample of cells from the subject with at least one inhibitor of LRP1 expression or activity, wherein a decrease in LRP1 expression or activity in the presence of the inhibitor as compared to the LRP1 expression or activity in the absence of the inhibitor identifies the inhibitor as useful for treating the disease. The sample of cells examined according to the present method can be obtained from the subject to be treated, or can be cells of an established ocular disease cell line or known ocular disease of the same type as that of the subject. In one aspect, the established cell line can be one of a panel of such cell lines, wherein the panel can include different cell lines of the same type of disease and/or different cell lines of different diseases associated with increase intraocular pressure. Such a panel of cell lines can be useful, for example, to practice the present method when only a small number of cells can be obtained from the subject to be treated, thus providing a surrogate sample of the subject's cells, and also can be useful to include as control samples in practicing the present methods.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy. In other embodiments, the biological sample of the present invention is a sample of bodily fluid, e.g., intraocular fluid, serum, and plasma.

As used herein “corresponding normal cells” means cells that are from the same organ and of the same type as the cells being examined. In one aspect, the corresponding normal cells comprise a sample of cells obtained from a healthy individual. Such corresponding normal cells can, but need not be, from an individual that is age-matched and/or of the same sex as the individual providing the cells being examined. In another aspect, the corresponding normal cells comprise a sample of cells obtained from an otherwise healthy portion of tissue of a subject having an ocular disorder.

Once disease is established and a treatment protocol is initiated, the methods of the invention may be repeated on a regular basis to evaluate whether the level of LRP1 expression or activity in the subject begins to approximate that which is observed in a normal subject. Alternatively, or in addition thereto, the methods of the invention may be repeated on a regular basis to evaluate whether symptoms associated with the particular ocular disease from which the subject suffers have been decreased or ameliorated. The results obtained from successive assays may be used to show the efficacy of treatment over a period ranging from several days to months to years. Accordingly, the invention is also directed to methods for monitoring a therapeutic regimen for treating a subject having an ocular disease. A comparison of the levels of LRP1 expression or activity and/or a comparison of the symptoms associated with the particular ocular disorder prior to and during therapy indicates the efficacy of the therapy. Therefore, one skilled in the art will be able to recognize and adjust the therapeutic approach as needed.

The total amount of a compound or composition to be administered in practicing a method of the invention can be administered to a subject as a single dose, either as a bolus or by infusion over a relatively short period of tune, or can be administered using a fractionated treatment protocol, in which multiple doses are administered over a prolonged period of time. One skilled in the art would know that the amount of the inhibitor of LRP1 expression or activity to treat ocular disorders in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

### EXAMPLES

The following Example is intended to illustrate but not limit the invention. In the Example, healthy subjects are used as opposed to models of glaucoma. This is intentional due to the fact that the majority of glaucoma models are mechanical and not representative of the physiological factors that lead to glaucoma in the vast majority of patients. Further, IOP reduction is largely agnostic to disease state, and reduction of IOP is the primary limiting factor in demonstration of clinical efficacy despite baseline levels. As such, the ability of an agent to reduce basal IOP is just as indicative of utility as reduction in a glaucomatous animal. Additionally, glaucoma models are highly variable and require large numbers of animals and substantial time to allow for RGC loss and subsequent demonstration of efficacy.

### Example 1

### Effect of RAP on IOP

Rat eyes are injected with 4 μl of an approximately 200 μM solution of RAP and intraocular pressure (IOP) is assessed at the time points indicated. Baseline measurements are acquired prior to injection. Rat eyes are also injected with C3 transferase (positive control) or vehicle (negative control). “Post” time-points are assessed approximately 60 minutes post-injections. “5 h” time-points are assessed approximately 5 hours post injections. “O/N” readings were taken the following morning, approximately 24 hours post injections.

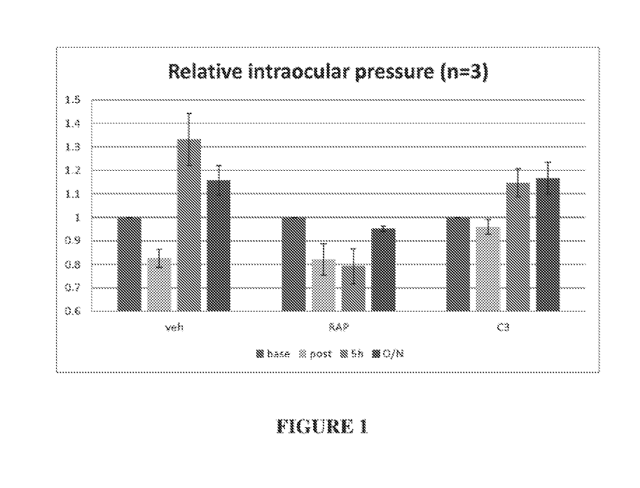

The results are shown in FIG. 1 as relative IOP to the base measurements. As shown in FIG. 1, RAP reduces IOP, especially at 5 hours post-injection and 24 hours post-injection, and is more effective than the positive control.

Although the invention has been described in detail above, it will be understood that modifications and variations are encompassed within the spirit and scope of the invention.

1. A method of treating an ocular disease comprising administering to a subject n need thereof a therapeutically effective amount of receptor associated protein (RAP), a derivative of RAP, a variant of RAP, or a fragment of RAP, thereby treating the ocular disease.

2. The method of claim 1, wherein the ocular disease is glaucoma or intraocular hypertension.

3. The method of claim 1, wherein administering comprises intravitreal injection.

4. (canceled)

5. The method of claim 1, wherein the subject s human.

6. The method of claim 1, wherein the administration of RAP, a derivative of RAP, a variant of RAP, or a fragment of RAP results in increased aqueous outflow of the vitreous humor of the eye.

7. The method of claim 1, wherein the administration comprises intravitreal delivery of a pharmaceutical composition that comprises a delivery vehicle and an expression vector that encodes the RAP, derivative of RAP, variant of RAP, or fragment of RAP.

8. The method of claim 7, wherein the expression vector is selected from the group consisting of an adenovirus vector, an adeno-associated virus vector, a herpes simplex virus-based vector, a lentivirus vector, and a plasmid vector.

9. A method of treating glaucoma comprising administering to a subject in need thereof a therapeutically effective amount of receptor associated protein (RAP), a derivative of RAP, a variant of RAP, or a fragment of RAP, thereby treating glaucoma.

10. The method of claim 9, wherein administering comprises intravitreal injection.

11. (canceled)

12. The method of claim 9, wherein the subject is human.

13. The method of claim 9, wherein the administration comprises intravitreal delivery of a pharmaceutical composition that comprises a delivery vehicle and an expression vector that encodes the RAP, derivative of RAP, variant of RAP, or fragment of RAP.

14. The method of claim 13, wherein the expression vector is selected from the group consisting of an adenovirus vector, an adeno-associated virus vector, a herpes simplex virus-based vector, a lentivirus vector, and a plasmid vector.

15. The method of claim 9, wherein the administration of RAP, a derivative of RAP, a variant of RAP, or a fragment of RAP results in increased aqueous outflow of the vitreous humor of the eve.

16. A method of reducing intraocular pressure in an eye of a subject comprising administering a RAP-conjugated agent, a RAP derivative-conjugated agent, a RAP variant-conjugated agent, or a RAP fragment-conjugated agent.

17. The method of claim 16, wherein the agent is a neurotrophic or neuroprotective agent.

18. The method of claim 17, wherein the neurotrophic or neuroprotective agent is selected from the group consisting of brain-derived neurotrophic factor (BDNF), nerve growth factor (NGF), neurotrophin-3/4 (NT-3/4), and ciliary neurotrophic factor (CNTF).

19. The method of claim 16, wherein administering comprises intravitreal injection.

20. (canceled)

21. The method of claim 16, wherein the subject is human.

22. The method of claim 16, wherein the administration comprises intravitreal delivery vector that encodes the RAP-conjugated agent, the RAP derivative-conjugated agent, the RAP variant-conjugated agent, or the RAP fragment-conjugated agent.

23. The method of claim 22, wherein the expression vector is selected from the group consisting of an adenovirus vector, an adeno-associated virus vector, a herpes simplex virus-based vector, a lentivirus vector, and a plasmid vector.

24. The method of claim 16, wherein the administration of the RAP-conjugated agent, RAP derivative-conjugated agent, RAP variant-conjugated agent, or RAP fragment-conjugated agent results in increased aqueous outflow of the vitreous humor of the eye.
